SEQUENCE LISTING (34 sequences)

SEQ ID NO: 1 - DNA, 84 bp, Artificial Sequence
  Synthetic polynucleotide
tgagtggggaactattggatgcattcgctcaaggggaaaaggagaaaggaagggtaaaagaagagagggaaagtaactttttaa

SEQ ID NO: 2 - DNA, 103 bp, Artificial Sequence
  Synthetic polynucleotide
tgagtggggaactattggatgcattcgctcaaggggaaaaggagaaaggaaggggaaaaggagaaaggaagggtaaaagaagagagggaaagtaactttttaa

SEQ ID NO: 3 - DNA, 70 bp, Artificial Sequence
  Synthetic polynucleotide
gacatgacagagctagtacagttctcattctgtgtgtgtgtatgtaaaggaagggtaacaggaaagagct

SEQ ID NO: 4 - DNA, 72 bp, Artificial Sequence
  Synthetic polynucleotide
gacatgacagagctagtacagttctcattctgtgtgtgtgtgtatgtaaaggaagggtaacaggaaagagct

SEQ ID NO: 5 - DNA, 74 bp, Artificial Sequence
  Synthetic polynucleotide
gacatgacagagctagtacagttctcattctgtgtgtgtgtgtgtatgtaaaggaagggtaacaggaaagagct

SEQ ID NO: 6 - DNA, 76 bp, Artificial Sequence
  Synthetic polynucleotide
gacatgacagagctagtacagttctcattctgtgtgtgtgtgtgtgtatgtaaaggaagggtaacaggaaagagct

SEQ ID NO: 7 - DNA, 78 bp, Artificial Sequence
  Synthetic polynucleotide
gacatgacagagctagtacagttctcattctgtgtgtgtgtgtgtgtgtatgtaaaggaagggtaacaggaaagagct

SEQ ID NO: 8 - DNA, 80 bp, Artificial Sequence
  Synthetic polynucleotide
gacatgacagagctagtacagttctcattctgtgtgtgtgtgtgtgtgtgtatgtaaaggaagggtaacaggaaagagct

SEQ ID NO: 9 - DNA, 82 bp, Artificial Sequence
  Synthetic polynucleotide
gacatgacagagctagtacagttctcattctgtgtgtgtgtgtgtgtgtgtgtatgtaaaggaagggtaacaggaaagagct

SEQ ID NO: 10 - DNA, 84 bp, Artificial Sequence
  Synthetic polynucleotide
gacatgacagagctagtacagttctcattctgtgtgtgtgtgtgtgtgtgtgtgtatgtaaaggaagggtaacaggaaagagct

SEQ ID NO: 11 - DNA, 86 bp, Artificial Sequence
  Synthetic polynucleotide
gacatgacagagctagtacagttctcattctgtgtgtgtgtgtgtgtgtgtgtgtgtatgtaaaggaagggtaacaggaaagagct

SEQ ID NO: 12 - DNA, 88 bp, Artificial Sequence
  Synthetic polynucleotide
gacatgacagagctagtacagttctcattctgtgtgtgtgtgtgtgtgtgtgtgtgtgtatgtaaaggaagggtaacaggaaagagct

SEQ ID NO: 13 - DNA, 90 bp, Artificial Sequence
  Synthetic polynucleotide
gacatgacagagctagtacagttctcattctgtgtgtgtgtgtgtgtgtgtgtgtgtgtgtatgtaaaggaagggtaacaggaaagagct

SEQ ID NO: 14 - DNA, 92 bp, Artificial Sequence
  Synthetic polynucleotide
gacatgacagagctagtacagttctcattctgtgtgtgtgtgtgtgtgtgtgtgtgtgtgtgtatgtaaaggaagggtaacaggaaagagct

SEQ ID NO: 15 - DNA, 94 bp, Artificial Sequence
  Synthetic polynucleotide
gacatgacagagctagtacagttctcattctgtgtgtgtgtgtgtgtgtgtgtgtgtgtgtgtgtatgtaaaggaagggtaacaggaaagagct

SEQ ID NO: 16 - DNA, 96 bp, Artificial Sequence
  Synthetic polynucleotide
gacatgacagagctagtacagttctcattctgtgtgtgtgtgtgtgtgtgtgtgtgtgtgtgtgtgtatgtaaaggaagggtaacaggaaagagct

SEQ ID NO: 17 - DNA, 98 bp, Artificial Sequence
  Synthetic polynucleotide
gacatgacagagctagtacagttctcattctgtgtgtgtgtgtgtgtgtgtgtgtgtgtgtgtgtgtgtatgtaaaggaagggtaacaggaaagagct

SEQ ID NO: 18 - DNA, 100 bp, Artificial Sequence
  Synthetic polynucleotide
gacatgacagagctagtacagttctcattctgtgtgtgtgtgtgtgtgtgtgtgtgtgtgtgtgtgtgtgtatgtaaaggaagggtaacaggaaagagct

SEQ ID NO: 19 - DNA, 102 bp, Artificial Sequence
  Synthetic polynucleotide
gacatgacagagctagtacagttctcattctgtgtgtgtgtgtgtgtgtgtgtgtgtgtgtgtgtgtgtgtgtatgtaaaggaagggtaacaggaaagagct

SEQ ID NO: 20 - DNA, 104 bp, Artificial Sequence
  Synthetic polynucleotide
gacatgacagagctagtacagttctcattctgtgtgtgtgtgtgtgtgtgtgtgtgtgtgtgtgtgtgtgtgtgtatgtaaaggaagggtaacaggaaagagct

SEQ ID NO: 21 - DNA, 106 bp, Artificial Sequence
  Synthetic polynucleotide
gacatgacagagctagtacagttctcattctgtgtgtgtgtgtgtgtgtgtgtgtgtgtgtgtgtgtgtgtgtgtgtatgtaaaggaagggtaacaggaaagagct

SEQ ID NO: 22 - DNA, 108 bp, Artificial Sequence
  Synthetic polynucleotide
gacatgacagagctagtacagttctcattctgtgtgtgtgtgtgtgtgtgtgtgtgtgtgtgtgtgtgtgtgtgtgtgtatgtaaaggaagggtaacaggaaagagct

SEQ ID NO: 23 - DNA, 110 bp, Artificial Sequence
  Synthetic polynucleotide
gacatgacagagctagtacagttctcattctgtgtgtgtgtgtgtgtgtgtgtgtgtgtgtgtgtgtgtgtgtgtgtgtgtatgtaaaggaagggtaacaggaaagagct

SEQ ID NO: 24 - DNA, 112 bp, Artificial Sequence
  Synthetic polynucleotide
gacatgacagagctagtacagttctcattctgtgtgtgtgtgtgtgtgtgtgtgtgtgtgtgtgtgtgtgtgtgtgtgtgtgtatgtaaaggaagggtaacaggaaagagct

SEQ ID NO: 25 - DNA, 114 bp, Artificial Sequence
  Synthetic polynucleotide
gacatgacagagctagtacagttctcattctgtgtgtgtgtgtgtgtgtgtgtgtgtgtgtgtgtgtgtgtgtgtgtgtgtgtgtatgtaaaggaagggtaacaggaaagagct

SEQ ID NO: 26 - DNA, 116 bp, Artificial Sequence
  Synthetic polynucleotide
gacatgacagagctagtacagttctcattctgtgtgtgtgtgtgtgtgtgtgtgtgtgtgtgtgtgtgtgtgtgtgtgtgtgtgtgtatgtaaaggaagggtaacaggaaagagct

SEQ ID NO: 27 - DNA, 118 bp, Artificial Sequence
  Synthetic polynucleotide
gacatgacagagctagtacagttctcattctgtgtgtgtgtgtgtgtgtgtgtgtgtgtgtgtgtgtgtgtgtgtgtgtgtgtgtgtgtatgtaaaggaagggtaacaggaaagagct

SEQ ID NO: 28 - DNA, 120 bp, Artificial Sequence
  Synthetic polynucleotide
gacatgacagagctagtacagttctcattctgtgtgtgtgtgtgtgtgtgtgtgtgtgtgtgtgtgtgtgtgtgtgtgtgtgtgtgtgtgtatgtaaaggaagggtaacaggaaagagct

SEQ ID NO: 29 - DNA, 21 bp, Artificial Sequence
  Synthetic polynucleotide
ggtgccaatatctggggaagg

SEQ ID NO: 30 - DNA, 20 bp, Artificial Sequence
  Synthetic polynucleotide
cccgtccacatccattttcc

SEQ ID NO: 31 - DNA, 493 bp, Artificial Sequence
  Synthetic polynucleotide
ggtgccaatatctggggaagggatgtgggagggggacagagagggactggggagtaaatgagtggggaactattggatgcattcgctcaaggggaaaaggagaaaggaagggtaaaagaagagagggaaagtaactttttaaaaaacaaagcaaggccaggcatggtagctcaagcctgtaatcccagcacaaggcaggaggattgcttgaggccaggaatttacgactagccccgggaacatagcaagaccagtctctacaaaactaaaaactaaatattagccaagggtagcacgtgcctatagtcccagctcctcaggaggctgaggtgggaggatcgtgtgagcccaggagatccaggctgcagtgagctatgatcgtatcactgtgtatcatgcccctgcagggtcagagcaagaccctctttctacaagaaaacaaacacaagcacaacaagaatatgaagaggtggggaaaatggatgtggacggg

SEQ ID NO: 32 - DNA, 23 bp, Artificial Sequence
  Synthetic polynucleotide
ccctccctgtctcttcttcatgg

SEQ ID NO: 33 - DNA, 20 bp, Artificial Sequence
  Synthetic polynucleotide
tgtggacccagcttctcagc

SEQ ID NO: 34 - DNA, 273 bp, Artificial Sequence
  Synthetic polynucleotide
ccctccctgtctcttcttcatggaagctgagtaccttctatgtatcaggcattacatatgcacctatagttcagttactcctcaaaataccctgtgagttattgccattttatagatgagaaaactgagtttcaaagacatgacagagctagtacagttctcattctgtgtgtgtgtgtgtgtgtgtgtgtgtgtgtgtgtatgtaaaggaagggtaacaggaaagagctggagaaacaagaacctgatctgggctgagaagctgggtccaca

(19) United States
(12) Patent Application Publication
(10) Pub. No.: US 2014/0037549 A1
(43) Pub. Date: Feb. 6, 2014

(54) GENETIC MARKERS FOR PROGNOSIS OF RHEUMATOID ARTHRITIS TREATMENT EFFICACY

(71) Applicants: Akiva Pinchas, Gedera (IL); Sarit Edelheit-Flohr, Tel Aviv (IL); Lily Bazak, Givatayim (IL); Zurit Levine, Herzlia (IL); Amir Toporik, Holon (IL); Ofer Levy, Moshav Mesilat Zion (IL); Tomer Zekharya, D.N. Shimshon (IL); Assaf Wool, Kiryat Ono (IL)

(72) Inventors: Akiva Pinchas, Gedera (IL); Sarit Edelheit-Flohr, Tel Aviv (IL); Lily Bazak, Givatayim (IL); Zurit Levine, Herzlia (IL); Amir Toporik, Holon (IL); Ofer Levy, Moshav Mesilat Zion (IL); Tomer Zekharya, D.N. Shimshon (IL); Assaf Wool, Kiryat Ono (IL)

(73) Assignee: COMPUGEN LTD., Tel Aviv (IL)

(21) Appl. No.: 13/985,468

(22) PCT Filed: Feb. 12, 2012
(86) PCT No.: PCT/IL12/00073

§ 371 Date: Oct. 17, 2013

Related U.S. Application Data
(60) Provisional application No. 61/443,255, filed on Feb. 16, 2011.

(51) Int. Cl. C12Q 1/68

(52) U.S. Cl. CPC C12Q 1/6883
USPC 424/9.2; 435/6.11; 506/9; 506/16

(57) ABSTRACT

The present invention discloses markers useful in predicting the responsiveness of a subject having, or at risk of developing rheumatoid arthritis (RA) to treatment with anti-TNF agents. Particularly, the present invention discloses the allelic configuration of the CD6 gene and/or the STXBP6 gene which is indicative of the degree of responsiveness to anti-TNFα treatment of RA.

### FIELD OF THE INVENTION

The present invention relates to genetic markers for predicting the responsiveness to treatment with anti tumor-necrosis-factor-α (TNFα) agents for rheumatoid arthritis and use thereof.

### BACKGROUND OF THE INVENTION

Rheumatoid arthritis (RA) is a chronic inflammatory disorder that affects synovial joints and has a prevalence of about 1% in the Western population. The inflammation is driven by overproduction, largely by macrophages, of the pro-inflammatory cytokines tumor-necrosis-factor-α (TNFα) and interleukin 1b (IL 1b). Autocrine and paracrine involvement of these cytokines within the joint space results in maintenance of the inflammatory response, leading to cartilage degradation and bone erosion.

The key role of TNFα in the inflammatory process of RA has led to the development of therapeutic strategies targeted to inhibit TNFα. The anti-TNFα drugs most commonly used in clinical practice are etanercept (Enbrel®), infliximab (Remicade®) and adalimumab (Humira®). Etanercept is a human, soluble, dimeric TNF type II receptor linked to an IgG1 Fc half that binds to and inactivates TNFα. The chimeric IgG1 monoclonal antibody infliximab and the complete humanized IgG1 monoclonal antibody adalimumab bind to TNFα with high affinity and thereby inactivate it. The therapeutic effect of these biological agents is achieved by blocking the potential interaction of TNFα with the accessory TNF cell-surface receptors. In this manner, an expanding array of drug therapy options for the treatment of rheumatoid arthritis in the clinic has been established over the past decade.

However, high costs, adverse drug events and unintentional concomitant immune suppression, leading to serious (opportunistic) infections, present limitations that might prevent the prescription of these biological drugs. For example, Bongartz et al. (Bongartz T et al. 2006 J Am Me. Assoc 295, 2275-2285) have provided evidence for a higher risk of serious infections and a dose-dependent, increased risk of malignancies in patients with RA who are treated with anti-TNF antibody therapy. Another limitation is that the treatment outcome of the TNF inhibitors remains insufficient in about 40-60% of patients with RA. Patients not responding to therapy can be divided to two groups: primary non-responders, which show lack of treatment efficacy from the first administration of an anti-TNF drug; and secondary non-responders showing an initial benefit from the treatment, but the effect is reduced within time.

The primary and secondary inefficacy of anti-TNF therapy in a significant percentage of RA patients, the adverse effects shown and the high cost of the anti-TNF blocking agents have driven the search for markers that can predict treatment outcome, including the search for genetic markers. Insight into the pharmacogenetics of anti-TNF therapy may facilitate the choice for the most suitable anti-TNF agent for an individual patient, and perhaps for the optimal dosing strategy.

Studies in the field of anti-TNF pharmacogenetics revealed genetic variation in several genes, particularly genes that can be directly linked to rheumatoid arthritis and/or its mechanism of action (review, for example, in Kooloos W M et al, 2007 Drug Disc 12(3/4), 125-131; Coene M J H et al., 2007 Pharmacogenomics 8(7), 761-773).

Several published studies reported biomarkers for predicting response to anti-TNFα treatment in rheumatoid arthritis patients. Criswell et al. describe specific shared epitope alleles in the HLA-DRB 1 region that are associated with better response to etanercept (Criswell L A et al. 2004 Arthritis Rheum 50(9), 2750-2756). Maxwell et al. (Maxwell J R et al. 2008 Hum. Mol. Genet. 17(22), 3532-3538), found two SNPs in the TNF gene which are associated with response to etanercept or infliximab. In a genome wide association study, Liu et al., found 16 SNPs that may be linked to response to anti TNFα treatment (Liu C et al. 2008 Mol. Med. 14(9-10), 575-581). Few studies have reported gene expression markers found in peripheral blood cells (Lequerre T et al. 2006 Arthritis Res Ther 8(4), R105; Sekiguchi N et al. 2008 Rheumatology (Oxford) 47(6), 780-788; Koczan D et al. 2008 Arthritis Res Ther 10(3), R50). In addition, Lindberg et al. (Lindberg J et al. 2006 Arthritis Res. Ther. 8(6), R179) found gene expression markers in synovial fluid. Other studies found that the expression level of several proteins in peripheral blood, including, for example Monocyte Chemoattractant Protein 1 (MCP1) and Epidermal Growth Factor (EGF), correlate with response to anti-TNFα treatment (Fabre S et al. 2008 Clin Exp. Immunol 153(2), 188-195; Hueber W et al. 2009 Arthritis Res Ther 11(3), 115).

U.S. patent Application Publication No. 20040009479 discloses methods of diagnosing or monitoring an autoimmune or chronic inflammatory disease in a patient by detecting the expression level of one or more genes or surrogates derived therefrom in the patient. Diagnostic oligonucleotides for diagnosing or monitoring chronic inflammatory disease, kits or systems containing the same are also described. The invention is particularly related to diagnosis of lupus erythematosis (SLE).

International Application Publication No. WO 2007/038501 discloses markers useful for diagnosing RA and for determining whether a subject with RA is likely to respond to TNF blockade therapy, particularly by determining whether the level of at least one marker is elevated in the subject compared to the corresponding average level in a sample of RA patients that are not responsive to TNF blockade therapy, wherein the markers are (a) transforming growth factor β-receptor 3 (TGFβR3) protein in serum; (b) interleukin-6 receptor (IL-6R) mRNA in peripheral blood cells; or (c) IL-6R protein in peripheral blood.

Additional approach for predicting the response of a patient to treatment with anti-TNF agent, particularly anti-TNFα antibody, is disclosed in International Application Publication No. WO 2008/142405. The method disclosed in that Application comprises exposing T cells present in an in vitro sample to an anti-TNFα therapy and determining whether regulatory T cells are induced in said sample of T cells. Induction of regulatory T cells indicates that the patient is likely to respond to treatment with said anti-TNFα therapy.

U.S. Application Publication No. 20060216707 discloses an array comprising oligo- or poly-nucleotide probes, particularly probes having the sequences of selective monocytic macrophagic genes, which are immobilized on a solid support. The array permits the diagnosis of rheumatoid arthritis and other chronic inflammatory diseases, a corresponding analysis of the efficacy of treatment and the monitoring of side-effects of the anti-tumor necrosis factor (TNF) therapy. The array is thus useful for the selection of an effective therapy for each patient with rheumatoid arthritis. The invention further relates to a nucleic acid array for the prognosis and development of novel anti-TNF type medicaments.

However, genetic markers and/or sets of such markers predicting the outcome of anti-TNF therapy with high specificity and sensitivity are still not available, and thus there is a recognized need for additional markers that answer the shortcomings of known markers.

### SUMMARY OF THE INVENTION

The present invention discloses a correlation between specific allelic gene variants and the response of rheumatoid arthritis patients to anti-TNFα treatment. Particularly, the present invention discloses the correlation of allelic variants of CD6, also known as T-cell differentiation antigen CD6, and of syntaxin binding protein 6 (STXBP6) with responsiveness to anti-TNFα rheumatoid arthritis therapies. Out of a number of genes known, to a certain degree, to be associated with rheumatoid arthritis (RA), the present invention now shows a particular allelic configuration of the CD6 and/or STXBP6 that is significantly associated with responsiveness to anti-TNFα treatment. This association of allelic variants and anti-TNFα response may be used as a diagnostic and/or prognostic tool. Patients having a genetic profile associated with a positive response to treatment with anti-TNFα agents may be beneficially treated with those agents. Patients having a genetic profile rendering them non-responsive may be treated with one or more alternative treatments of RA. In addition, identifying patient populations comprising the responsive or non-responsive allelic profile is highly beneficial for drug development, assigning the adequate population for examining anti-TNFα-based drugs and thus optimizing the outcome of such studies.

Thus, according to one aspect, the present invention provides a method for determining responsiveness to anti-TNFα therapy in a mammal having, or at risk of developing rheumatoid arthritis comprising determining the allelic configuration of at least one of CD6 gene; STXBP6 gene; or a combination thereof, wherein the allelic configuration of the CD6 gene comprises a variant allele having an insertion of nucleic acids and a wild-type allele lacking the insertion and wherein the allelic configuration of the STXBP6 gene comprises multiple alleles having between 5 and 30 TG repeats.

According to certain embodiments, the insertion of the CD6 variant allele is at position 60,542,552 on chromosome 11 (NCBI build 36). According to one embodiment, the insertion is of 19 base pairs. According to certain typical embodiments, the nucleic acid sequence of the CD6 variant allele comprising the insertion comprises the nucleic acid sequence set forth in SEQ ID NO:2 and the nucleic acid sequence of the wild type allele lacking said insertion comprises the nucleic acid sequence set forth in SEQ ID NO:1.

According to other embodiments, the STXBP6 gene configuration comprises at least one short allele comprising from 5 to 21 TG repeats replacing a segment located at positions 24,503,694-24,503,727 on chromosome 14 (NCBI build 36), and at least one long STXBP6 allele comprising from 22 to 30 TG repeats at the same location. According to typical embodiments, the nucleic acid sequence of the short STXBP6 allele comprises the nucleic acid sequence set forth in any one of SEQ ID NOs:3-19 and of the long alleles comprises the nucleic acid sequence set forth in any one of SEQ ID NOs:20-28.

According to certain typical embodiments, the presence of at least one CD6 variant allele and/or two STXBP6 short alleles is indicative of good responsiveness to anti-TNFα-treatment for RA.

According to other typical embodiments, the presence of two CD6 wild type alleles and/or at least one long STXBP6 allele is indicative of moderate or non-responsiveness to anti-TNFα-treatment for RA.

The allelic configuration may be determined using any method as is known to a person skilled in the art. According to certain embodiments, the allelic configuration is determined using polynucleotide sequencing, fragment analysis, multiplex ligation-dependent, probe amplification (MLPA), microarray analysis, allelic-specific amplification and/or hybridization and the like, each being a separate embodiment of the invention.

According to certain embodiments, the anti-TNFα treatment is selected from the group consisting of, but not limited to, etanercept, infliximab, adalimumab, certolizumab, Cimzia (certolizumab pegol), golimumab (also known as Simponi®)).

According to additional aspect, the present invention provides a method for determining the effectiveness of anti-TNFα therapy in a population of subjects having, or at risk of developing RA, comprising: determining the allelic configuration of at least one of CD6 gene; STXBP6 gene; or a combination thereof; selecting subjects having at least one CD6 variant allele; two short STXBP6 allele or a combination thereof; determining the RA status of the selected subjects; administering the therapy at least once to said subject; and determining the RA status after administration of said therapy; wherein improvement in the RA status is indicative of a positive effect of said therapy.

According to certain embodiments, the method further comprises determining the presence of at least one additional marker known or predicted to correlate with a response to anti-TNFα treatment in rheumatoid arthritis patients. According to one embodiment, the additional marker is selected from, but not limited to, specific shared epitope alleles in the HLA-DRB1 region associated with better response to etanercept (Criswell L A, et al., ibid) and SNPs in the TNF gene (Maxwell J R, et al., ibid).

According to a further aspect, the present invention provides a kit comprising at least one oligonucleotide capable of specifically detecting the allelic configuration of at least one of CD6 gene and STXBP6 gene.

According to certain embodiments, the oligonucleotide is capable of specifically distinguishing between the CD6 variant and wild-type alleles disclosed herein. According to other embodiments, the oligonucleotide is capable of distinguishing between the STXBP6 short and long alleles disclosed herein. According to one embodiment, the oligonucleotide specifically hybridizes to a polynucleotide having a nucleic acid sequence selected from the group consisting of SEQ ID NOs:1-28.

According to other embodiments, the kit comprises a primer pair capable of specifically amplifying a polynucleotide having a nucleic acids sequence selected from the group consisting of SEQ ID NOs:1-28.

According to certain embodiments, the primer pair comprises a pair of oligonucleotides having the nucleic acid sequence selected from the group consisting of SEQ ID NOs: 29-30 and 32-33, corresponding to the CD6 and STXBP6 alleles, respectively.

According to one embodiment, the primer pair comprising the oligonucleotide having the nucleic acid sequence set forth in SEQ ID NOs:29-30 amplifies a polynucleotide having the nucleic acid sequence set forth in SEQ ID NO:31. According to another embodiment, the primer pair comprising the oligonucleotide having the nucleic acid sequence set forth in SEQ ID NOs:32-33 amplifies a polynucleotide having the nucleic acid sequence set forth in SEQ ID NO:34.

According to typical embodiments, the kit comprises reagents for employing an assay for detecting the allelic configuration of at least one of CD6 gene and STXBP6 gene as disclosed herein.

According to one embodiment, the reagents are for genotyping analysis. As used herein, “genotyping analysis” refers to any analysis that provides a measurement of the genetic variation between members of a species. According to typical embodiments, the genotyping analysis is performed employing a NAT-based assay, selected from the group consisting of hybridization-based methods, enzyme-based methods, other post-amplification methods based on physical properties of DNA, and sequencing technologies. According to further typical embodiments, the NAT-based assay is selected from the group consisting of a PCR, Real-Time PCR, LCR, Tetra-primer ARMS-PCR, Cycling Probe Reaction, Multiplex ligation-dependent probe amplification (MLPA), Fragment analysis, Dynamic allele-specific hybridization (DASH), molecular beacons, Branched DNA, RFLP analysis, Single-Strand Conformation Polymorphism analysis, Dideoxy Fingerprinting, Microarrays, Fluorescent in situ Hybridization, Comparative Genomic Hybridization, Flap endonuclease (FEN)-based assays, Invader assay, Serial Invasive Signal Amplification Reaction (SISAR), primer extension assays using a wide range of detection techniques that include MALDI-TOF Mass spectrometry and ELISA-like methods, 5′-nuclease activity used in Taqman assay, oligonucleotide ligase assay, temperature gradient gel electrophoresis (DGGE/TGGE), temperature gradient capillary electrophoresis (TGCE), denaturing high performance liquid chromatography (DHPLC), and High-Resolution Melting analysis.

Other objects, features and advantages of the present invention will become clear from the following description and drawings.

### BRIEF DESCRIPTION OF THE FIGURES

FIG. 1 shows the alignment of the two CD6 alleles. The upper sequence shows the wild type allele and the lower sequence shows the variant allele comprising the insertion.

FIG. 2 shows the STXBP6 long-alleles distribution in samples obtained from patient population comprising good, moderate and non-responders to anti-TNFα therapy.

### DETAILED DESCRIPTION OF THE INVENTION

The present invention shows for the first time the association of allelic variants of the CD6 gene and/or the STXBP6 gene with clinical response of patients having rheumatoid arthritis to anti-TNFα therapy. These alleles can be used as assessment markers for the responsiveness of a subject having rheumatoid arthritis (RA), or suspected to develop RA, to anti-TNFα based therapy. In addition, these markers can be used to enhance the efficacy of anti-TNFα drug development by selecting the most suitable RA patient population, i.e. patients who are responsive to the anti-TNFα therapy.

According to certain aspects, the present invention discloses two such markers. The first marker is a 19 bp insertion which may affect the splicing of CD6 (T-cell differentiation antigen CD6). The second is a 2 bp repeat (microsatellite) adjacent to a transcription factor binding site located in an intron of STXBP6 (syntaxin binding protein 6-amisyn). This variation may have a direct effect on the expression of the gene or may have an effect on a different transcription product in the region.

### DEFINITIONS

As used herein, the term “gene” has its meaning as understood in the art. In general, a gene is taken to include gene regulatory sequences (e.g. promoters, enhancers, etc.) and/or intron sequences, in addition to coding sequences (open reading frames) and untranslated regions (UTRs). It is to be further appreciated that definitions of “gene” include references to nucleic acids that do not encode proteins but rather encode functional RNA molecules such as microRNAs (miRNAs), tRNAs, etc.

The term “allele” as used herein refers to one of the different forms of a gene or DNA sequence that can exist at a single locus within the genome.

As used herein, the term “marker” refers to a nucleic acid fragment which may be present or absent in the genome of a subject that have rheumatoid arthritis (RA) or at risk to develop RA and may be correlated with response to anti-TNFα therapy for RA.

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modifications, including, for example, analogous linking groups, purine, pyrimidines, and sugars. However, the polynucleotides of the invention are preferably comprised of greater than 50% conventional deoxyribose nucleotides, and most preferably greater than 90% conventional deoxyribose nucleotides. The polynucleotide sequences of the invention may be prepared by any known method, including synthetic, recombinant, ex vivo generation, or a combination thereof, as well as utilizing any purification methods known in the art.

The term “primer” refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 12 to 30 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified.

The term “probe” or “hybridization probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified by hybridization. “Probes” or “hybridization probes” are nucleic acids capable of binding in a base-specific manner to a complementary strand of nucleic acid. Such probes include peptide nucleic acids, as described in Nielsen et al. (1991. Science 254:1497-1500). Hybridizations can be performed under “stringent conditions”, for example, at a salt concentration of no more than 1M and a temperature of at least 25° C. For example, conditions of 5×SSPE 750 mM NaCl, 50 mM NaPhosphate, 5 mM EDTA, pH 7.4 and a temperature of 25° C. to 30° C. are suitable for allele-specific probe hybridizations. Although this particular buffer composition is offered as an example, one skilled in the art could easily substitute other compositions of equal suitability.

The term “sequencing” as used herein means a process for determining the order of nucleotides in a nucleic acid. A variety of methods for sequencing nucleic acids are well known in the art. Such sequencing methods include the Sanger method of dideoxy-mediated chain termination as described, for example, in Sanger et al. 1977. Proc Natl Acad Sci 74:5463, which is incorporated herein by reference (see, also, “DNA Sequencing” in Sambrook et al. (eds.), Molecular Cloning: A Laboratory Manual (Second Edition), Plainview, N.Y.: Cold Spring Harbor Laboratory Press (1989), which is incorporated herein by reference). A variety of polymerases including the Klenow fragment of E. coli DNA polymerase I; Sequenase™ (T7 DNA polymerase); Taq D A polymerase and Amplitaq can be used in enzymatic sequencing methods. Well known sequencing methods also include Maxam-Gilbert chemical degradation of DNA (see Maxam and Gilbert, Methods Enzymol. 65:499 (1980)), which is incorporated herein by reference, and “DNA Sequencing” in Sambrook et al., supra, 1989). One skilled in the art recognizes that sequencing is now often performed with the aid of automated methods.

“Odds ratio” or “OR” is used herein to summarize the performance of a marker in predicting a response. OR is defined generally as the ratio of the odds of having a good response when the marker is positive to the odds of having a good response when the marker is negative. OR is defined herein according to the following equation:

$$O R = \frac{\frac{{p}_{1}}{1 - {p}_{1}}}{\frac{{p}_{2}}{1 - {p}_{2}}}$$

Where p₁ is the probability of good response when the marker is positive and p₂ is the probability of good response when the marker is negative.

### Preferred Mode of Carrying the Invention

Amplicon CGEN-40002 CD6 gene

CD6, also known as T-cell differentiation antigen CD6, is a cell surface receptor belonging to the scavenger receptor cysteine-rich (SRCR) protein superfamily (SRCRSF). It specifically binds activated leukocyte cell adhesion molecule (ALCAM, CD166), a member of the immunoglobulin (Ig) superfamily (IgSF).

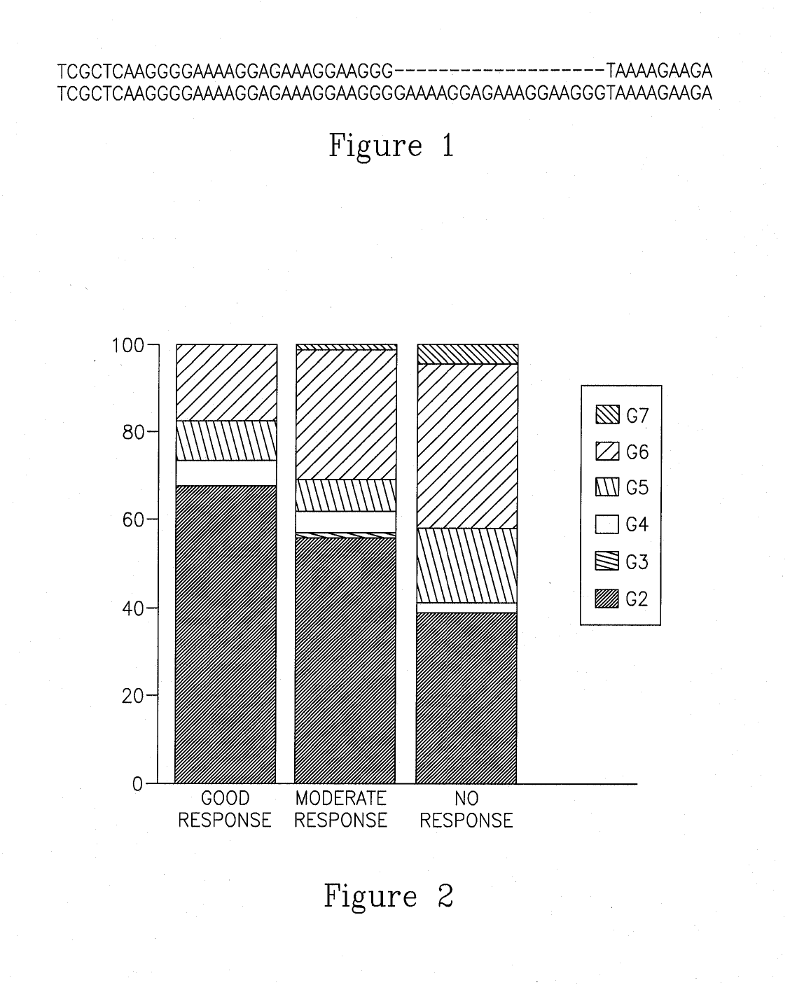

The CGEN-40002 amplicon represents a 19 bp insertion within the CD6 gene on chromosome 11. The variation can be found in version 130 of dbSNP, entry rs55799216. The genomic location of the insertion is at position 60,542,552 on chromosome 11 (NCBI build 36), the sequence alignment of the two alleles is shown in FIG. 1.

The inserted segment in the long allele is a copy of the preceding 19 bp of the short allele. The variation lies in the last intron of the gene, about 100 bp downstream of the end of the preceding exon. This exon is alternative according to RNA evidence, and there is also RNA evidence of intron retention for this intron, both in human and in chimpanzee. It is to be noted that the two human ESTs crossing this exon-intron junction (accession numbers BQ932452 and CR978174) contain the 19 bp insertion. It is possible that the variation has an influence on the splicing pattern of the gene.

The linkage of CD6 to rheumatoid arthritis has been previously disclosed, and antibodies to CD6 are under development as a treatment of rheumatoid arthritis (“Thomson Pharma” drug database: T-1 h-mAb a humanized anti-CD6 monoclonal antibody by Biocon; or T1 a murine anti-CD6 monoclonal antibody developed by The Center of Molecular Immunology in Cuba). However, the linkage of CD6 to responsiveness to anti-TNFα therapy is disclosed in the present invention for the first time.

### Amplicon CGEN-40003-STXBP6 Gene

Syntaxin-binding protein 6 (STXBP6) or Amisyn is a protein that regulates the formation of Soluble N-Ethylmaleimide-Sensitive Factor Attachment Protein Receptor (“SNARE”; Scales et al. J Biol. Chem. 2002 Aug. 2; 277(31):28271-9). The STXBP6 gene was shown, among many others, to be differentially expressed between responders and non-responders to anti-TNFα treatment (Lindberg et. al Arthritis Research & Therapy 2006, 8:R179). However, the gene was not identified as a promising candidate for predicting responsiveness, and furthermore, no particular variant was allocated to be significantly correlated with responsiveness, as disclosed in the present invention for the first time.

The CGEN-40003 amplicon represents a 2 bp short repeat or microsatellite within the STXBP6 gene on chromosome 14. The variation lies within a very long intron, approximately 10 Kbp away from the closest exon. This microsatellite is represented in dbSNP version 130 by several entries, for example rs10630160, rs34132743, rs35668825. The reference genome contains 17 TG repeats in positions 24,503,694-24,503,727, and dbSNP describes alleles with 1-9 additional TG repeats.

Adjacent to the variation's location is a highly conserved genomic region 250 bp long, with a high ESPERR (evolutionary and sequence pattern extraction through reduced representations) score signifying a potential functional element in the genome, and a possible transcription binding site for PPAR (Peroxisome proliferator-activated receptor) gamma. The variation may have a direct effect on the transcription of the STXBP6 gene. There may also be a different expressed sequence affected directly by the variation. One EST (AI274296) aligns to the genome 200 bp downstream of the variation and the Affymetrix exon chip contains a probe in this region. The expression profile for this probe was compared across several experiments (available on GEO) with the profile of several probes for the STXBP6 exons, and a lower signal with a Pearson correlation in the range of 0.6-0.7 was found. Without wishing to be bound by any specific theory or mechanism of action, this may indicate that the region is transcribed in connection with the gene, but not as frequently as the gene. Another possibility is that the variation is not causative but correlated with another variation in the region.

### General Methods of the Invention

The present invention discloses the association between certain allelic polynucleotides and the responsiveness of subject having RA to a treatment with drug(s) directed to block TNFα activity.

Any method as is known in the art for identifying the presence/absence of a certain allele may be used according to the teachings of the present invention.

According to typical embodiments, the detection of a nucleic acid of interest in a biological sample is effected by genotyping analysis, preferably by NAT-based assays, which involve nucleic acid amplification technology selected from, but not limited to, hybridization-based methods, enzyme-based methods, other post-amplification methods based on physical properties of DNA, and sequencing technologies. Amplification of a selected, or target, nucleic acid sequence may be carried out by a number of suitable methods. See generally Kwoh et al., 1990, Am. Biotechnol. Lab. 8, 14. Numerous amplification techniques have been described and can be readily adapted to suit particular needs of a person of an ordinary skill in the art. Non-limiting examples of amplification techniques include polymerase chain reaction (PCR), ligase chain reaction (LCR), tetra-primer ARMS PCR, strand displacement amplification (SDA), transcription-based amplification, the q3 replicase system and NASBA (Kwoh et al., 1989 Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, Molecular Cloning—A Laboratory Manual, 2nd Edition, CSH Laboratories).

The nucleic acid (i.e. DNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, ibid; Ausubel et al., 1989, in Current Protocols in Molecular Biology, John Wiley & Sons Inc., N.Y.).

Polymerase Chain Reaction (PCR): The polymerase chain reaction (PCR), as described in U.S. Pat. Nos. 4,683,195 and 4,683,202 to Mullis and Mullis et al. is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation and ligation amplify a short segment of DNA. LCR has also been used in combination with PCR to achieve enhanced detection of single-base changes: see for example PCT Publication No. WO9001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Tetra primer ARMS PCR reaction employs two pairs of primers to amplify two alleles in one PCR reaction. The primers are designed such that the two primer pairs overlap at a SNP location but each match perfectly to only one of the possible SNPs. As a result, if a given allele is present in the PCR reaction, the primer pair specific to that allele will produce product but not to the alternative allele with a different SNP. The two primer pairs are also designed such that their PCR products are of a significantly different length allowing for easily distinguishable bands by gel electrophoresis. In examining the results, if a genomic sample is homozygous, then the PCR products that result will be from the primer which matches the SNP location to the outer, opposite strand primer as well from the two opposite, outer primers. If the genomic sample is heterozygous, then products will result from the primer of each allele to their respective outer primer counterparts as well as from the two opposite, outer primers.

Many applications of nucleic acid detection technologies, such as in studies of allelic variation according to the teachings of the present invention, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may be carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

Another method for detection of genomic insertions or deletions is Fragment analysis, based on measuring the length of PCR products. One of the primers used for PCR is labeled (e.g. dye or fluorescent labels). The PCR products then go through capillary electrophoresis where the size of the product determines when the product is released. The release time is detected using the labeled primer. Standards of known length are used to calibrate the length measurement which is accurate up to two nucleotides. The product length difference caused by a genomic insertion or deletion within the product can thus be detected. Heterozygote samples that contain both a short and a long product in the same genomic location can also be detected using this method.

The detection of at least one sequence change according to certain embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Dynamic allele-specific hybridization (DASH), Denaturing High Performance Liquid Chromatography (DHPLC), Single-Strand Conformation Polymorphism (SSCP) analysis, primer extension, Dideoxy fingerprinting (ddF), MLPA, DASH, Molecular beacons, or High Resolution Melting analysis.

Multiplex ligation-dependent probe amplification (MLPA) is a variation of the polymerase chain reaction that permits multiple targets to be amplified with only a single primer pair. Each probe consists of a two oligonucleotides which recognize adjacent target sites on the DNA. One probe oligonucleotide contains the sequence recognized by the forward primer, the other the sequence recognized by the reverse primer. Only when both probe oligonucleotides are hybridized to their respective targets, can they be ligated into a complete probe. The advantage of splitting the probe into two parts is that only the ligated oligonucleotides, but not the unbound probe oligonucleotides, are amplified. If the probes were not split in this way, the primer sequences at either end would cause the probes to be amplified regardless of their hybridization to the template DNA, and the amplification product would not be dependent on the number of target sites present in the sample DNA. Each complete probe has a unique length, so that its resulting amplicons can be separated and identified by (capillary) electrophoresis. This avoids the resolution limitations of multiplex PCR. Since the forward primer used for probe amplification is fluorescently labeled, each amplicon generates a fluorescent peak which can be detected by a capillary sequencer. Comparing the peak pattern obtained on a given sample with that obtained on various reference samples, the relative quantity of each amplicon can be determined. This ratio is a measure for the ratio in which the target sequence is present in the sample DNA. MLPA can successfully and easily determine the relative copy number of all exons within a gene simultaneously with high sensitivity. For detailed description of MLPA Probe preparation and MLPA analysis, please refer to Schouten et al (Nucleic Acids Research, 2002, Vol. 30, No. 12 e57).

Dynamic allele-specific hybridization (DASH) genotyping takes advantage of the differences in the melting temperature in DNA that results from the instability of mismatched base pairs. The process can be vastly automated and encompasses a few simple principles. In the first step, a genomic segment is amplified and attached to a bead through a PCR reaction with a biotinylated primer. In the second step, the amplified product is attached to a streptavidin column and washed with NaOH to remove the unbiotinylated strand. An allele specific oligonucleotide is then added in the presence of a molecule that fluoresces when bound to double-stranded DNA. The intensity is then measured as temperature is increased until the Tm can be determined. A SNP will result in a lower than expected Tm (Howell et al. 1999, Nature Biology, 1:87-88). Because DASH genotyping is measuring a quantifiable change in Tm, it is capable of measuring all types of mutations, not just SNPs. Other benefits of DASH include its ability to work with label free probes and its simple design and performance conditions.

SNP detection through Molecular beacons makes use of a specifically engineered single-stranded oligonucleotide probe, designed such that there are complementary regions at each end and a probe sequence located in between. This design allows the probe to take on a hairpin, or stem-loop, structure in its natural, isolated state. Attached to one end of the probe is a fluorophore and to the other end a fluorescence quencher. Because of the stem-loop structure of the probe, the fluorophore is in close proximity to the quencher, thus preventing the molecule from emitting any fluorescence. The molecule is also engineered such that only the probe sequence is complementary to the genomic DNA that will be used in the assay (Abravaya et al. 2003, Clin Chem Lab Med 2003; 41(4):468-474). If the probe sequence of the molecular beacon encounters its target genomic DNA during the assay, it will anneal and hybridize. Because of the length of the probe sequence, the hairpin segment of the probe will denatured in favor of forming a longer, more stable probe-target hybrid. This conformational change permits the fluorophore and quencher to be free of their tight proximity due to the hairpin association, allowing the molecule to fluoresce. If on the other hand, the probe sequence encounters a target sequence with as little as one non-complementary nucleotide, the molecular beacon will preferentially stay in its natural hairpin state and no fluorescence will be observed, as the fluorophore remains quenched. The unique design of these molecular beacons allows for a simple diagnostic assay to identify SNPs at a given location. If a molecular beacon is designed to match a wild-type allele and another to match a mutant of the allele, the two can be used to identify the genotype of an individual. If only the first probe's fluorophore wavelength is detected during the assay then the individual is homozygous to the wild type. If only the second probe's wavelength is detected then the individual is homozygous to the mutant allele. Finally, if both wavelengths are detected, then both molecular beacons must be hybridizing to their complements and thus the individual must contain both alleles and be heterozygous.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism (RFLP) analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes has 4 to 6 base-pair recognition sequences, and cleaves too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs) can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic nobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

Flap endonuclease (FEN) is an endonuclease that catalyzes structure-specific cleavage. This cleavage is highly sensitive to mismatches and can be used to interrogate SNPs with a high degree of specificity (Olivier 2005). In the basic Invader assay, a FEN called cleavase is combined with two specific oligonucleotide probes, that together with the target DNA can form a tripartite structure recognized by cleavase. The first probe, called the Invader oligonucleotide is complementary to the 3′ end of the target DNA. The last base of the Invader oligonucleotide is a non-matching base that overlaps the SNP nucleotide in the target DNA. The second probe is an allele-specific probe which is complementary to the 5′ end of the target DNA, but also extends past the 3′ side of the SNP nucleotide. The allele-specific probe will contain a base complementary to the SNP nucleotide. If the target DNA contains the desired allele, the Invader and allele-specific probes will bind to the target DNA forming the tripartite structure. This structure is recognized by cleavase, which will cleave and release the 3′ end of the allele-specific probe. If the SNP nucleotide in the target DNA is not complementary allele-specific probe, the correct tripartite structure is not formed and no cleavage occurs. The Invader assay is usually coupled with fluorescence resonance energy transfer (FRET) system to detect the cleavage event. In this setup, a quencher molecule is attached to the 3′ end and a fluorophore is attached to the 5′ end of the allele-specific probe. If cleavage occurs, the fluorophore will be separated from the quencher molecule generating a detectable signal. Only minimal cleavage occurs with mismatched probes making the Invader assay highly specific. However, in its original format, only one SNP allele could be interrogated per reaction sample and it required a large amount of target DNA to generate a detectable signal in a reasonable time frame. Several developments have extended the original Invader assay. By carrying out secondary FEN cleavage reactions, the Serial Invasive Signal Amplification Reaction (SISAR) allows both SNP alleles to be interrogated in a single reaction. SISAR Invader assay also requires less target DNA, improving the sensitivity of the original Invader assay.

Primer extension is a two step process that first involves the hybridization of a probe to the bases immediately upstream of the SNP nucleotide followed by a ‘mini-sequencing’ reaction, in which DNA polymerase extends the hybridized primer by adding a base that is complementary to the SNP nucleotide. This incorporated base is detected and determines the SNP allele (Goelet et al. 1999; Syvanen 2001). Because, primer extension is based on the highly accurate DNA polymerase enzyme, the method is generally very reliable. Primer extension is able to genotype most SNPs under very similar reaction conditions making it also highly flexible. The primer extension method is used in a number of assay formats. These formats use a wide range of detection techniques that include MALDI-TOF Mass spectrometry and ELISA-like methods (Rapley & Harbron 2004, Molecular Analysis and Genome Discovery. Chichester. John Wiley & Sons Ltd. 388p).

Generally, there are two main approaches which use the incorporation of either fluorescently labeled dideoxynucleotides (ddNTP) or fluorescently labeled deoxynucleotides (dNTP). With ddNTPs, probes hybridize to the target DNA immediately upstream of SNP nucleotide, and a single, ddNTP complementary to the SNP allele is added to the 3′ end of the probe (the missing 3′-hydroxyl in didioxynucleotide prevents further nucleotides from being added). Each ddNTP is labeled with a different fluorescent signal allowing for the detection of all four alleles in the same reaction. With dNTPs, allele-specific probes have 3′ bases which are complementary to each of the SNP alleles being interrogated. If the target DNA contains an allele complementary to the probe's 3′ base, the target DNA will completely hybridize to the probe, allowing DNA polymerase to extend from the 3′ end of the probe. This is detected by the incorporation of the fluorescently labeled dNTPs onto the end of the probe. If the target DNA does not contain an allele complementary to the probe's 3′ base, the target DNA will produce a mismatch at the 3′ end of the probe and DNA polymerase will not be able to extend from the 3′ end of the probe. The benefit of the second approach is that several labeled dNTPs may get incorporated into the growing strand, allowing for increased signal. However, DNA polymerase in some rare cases can extend from mismatched 3′ probes giving a false positive result (Rapley & Harbron 2004).

A different approach is used by Sequenom's iPLEX SNP genotyping method, which uses a MassARRAY mass spectrometer. Extension probes are designed in such a way that 40 different SNP assays can be amplified and analyzed in a PCR cocktail. The extension reaction uses ddNTPs as above, but the detection of the SNP allele is dependent on the actual mass of the extension product and not on a fluorescent molecule. This method is for low to medium high throughput, and is not intended for whole genome scanning.

The flexibility and specificity of primer extension make it amenable to high throughput analysis. Primer extension probes can be arrayed on slides allowing for many SNPs to be genotyped at once. Broadly referred to as arrayed primer extension (APEX), this technology has several benefits over methods based on differential hybridization of probes. Comparatively, APEX methods have greater discriminating power than methods using this differential hybridization, as it is often impossible to obtain the optimal hybridization conditions for the thousands of probes on DNA microarrays (usually this is addressed by having highly redundant probes). However, the same density of probes cannot be achieved in APEX methods, which translates into lower output per run (Rapley & Harbron 2004, ibid).

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples from a population of subjects having RA or at risk of developing RA.

### Kits

The present invention also provides a kit comprising one or more, oligonucleotides capable of hybridizing to, or adjacent to, polymorphic sites in the CD6 or STXBP6 gene as disclosed in the present invention. The oligonucleotide(s) may be provided in solid form, in solution or attached on a solid carrier such as a DNA microarray. In addition, the kit may provide detection means, containers comprising solutions and/or enzymes and a manual with instructions for use.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Experimental Setup

The genotyping experiment contained 213 amplicons and 237 DNA samples.

### Clinical Data

The responsiveness to the anti-TNFα treatment was measured using the disease activity score for 28 joints (DAS28) before and after treatment, and the European League Against Rheumatism (EULAR) response criteria. DAS28 consists of four parameters combined in a mathematical formula to define the DAS28 score. The parameters include (1) counting the number of swollen joints (out of 28); (2) counting the number of tender joints (out of 28); (3) measuring ESR (erythrocyte sedimentation rate) and (4) general health assessment.

Good responsiveness is defined by a DAS28 score after treatment which is at least 1.2 lower than the DAS28 score before treatment and having a value of at most 3.2. Non-responsiveness is defined by either (1) a DAS28 score after treatment at most 0.6 lower than the DAS28 score before treatment irrespective of the DAS28 score value, or (2) a DAS28 score after treatment which is at most 1.2 lower than the DAS28 score before treatment and having a value of at least 5.1. Moderate responsiveness is defined by either (1) a DAS28 score after treatment between 0.6 and 1.2 lower than the DAS28 score before treatment, having a value of at most 5.1, or (2) a DAS28 score after treatment at least 1.2 lower than the DAS28 score before treatment, having a value of at least 3.2.

The clinical parameters available were:

1. Gender

2. Anti-TNF drug: infliximab, adalimumab or etanercept.

3. DAS28 before treatment.

4. DAS28 score after treatment.

5. Duration between DAS28 measurements. Typically, the duration of the treatment was 26 weeks.

6. Response classification according to the European League Against Rheumatism (EULAR): good, moderate or none.

Of the genotyped samples, 68 samples were of subjects showing good response, 81 of subjects showing moderate response and 88 of subject showing no response.

### Amplicons

Two methods were used for amplicon genotyping. 122 amplicons were genotyped using sequencing and 91 were genotyped using fragment analysis. Of the 213 available amplicons, two (2) failed genotyping, and another amplicon turned out to be non-variable. There were 71 amplicons with two alleles (bi-allelic) and 139 with three or more alleles (multi-allelic). Unexpectedly, of this large number of amplicons, only two were shown to stand significant statistical analyses as differentiating between responsiveness and non-responsiveness of RA patients to anti-TNFα treatment.

### Preparations for Analysis

In order to maximize the probability of discovering a response marker the genotypes of good responders and non responders were compared, excluding the moderate response group in the initial analysis. The tests planned divided the alleles of an amplicon into two groups, and then either the dominant or the recessive model was used for these groups. A 2×2 table of (genotype groups)×(good/no response) was then formed and the p-value was calculated using a Fisher exact test.

Two types of allele grouping were used: all alleles with length smaller or larger than a defined value, or one allele vs. all others. For bi-allelic amplicons there is only one allele grouping possible, one allele vs. the other. There are two tests possible in this case since the recessive and dominant models for one allele are the same as the dominant and recessive models for the other allele, respectively. For multi-allelic amplicons more tests are possible. Possible tests were considered only if the minimal genotype group size was at least 10% of the total number of samples with genotypes for this amplicon.

The clinical data enabled finding the number of test (Ntest) to be performed for each amplicon. There were 202 amplicons for which Ntest was at least one, hence the number of markers (Nmarker) is 202. The type I error threshold for any test for a certain amplicon is 0.05/(Nmarker×Ntest).

About one third of the patients have moderate EULAR response to treatment. The basic assumption employed was that for a marker correlating to a response, there is a distribution of the response for the group of good response, a distinctly different distribution of the response for the group of no response, and the moderate responders are likely to be in the middle with contributions from both genotypic groups. Therefore adding the moderate response samples to either the good or the non responders is likely to reduce the significance for a good marker. On the other hand, combining the samples of moderate response subjects increases the size of the population tested, making it possible for less extreme distributions to reach significance. Therefore for each test without the samples of moderate responders adding the moderate responders to either the good or the non responders was considered.

### Example 1

### CD6 Markers: CGEN-40002

The amplicon identified within the CD6 gene is bi-allelic, the wild-type allele (comprising SEQ ID NO:1) is represented by an amplicon length of 493 nucleotides SEQ ID NO:31) and the variant allele (comprising SEQ ID NO:2), represented by an amplicon length of 512 nucleotides. The genotype distribution is shown in Table 1:

TABLE 1
Genotype distribution of the CD6 amplicon

| Allele | WT | Variant |
|---|---|---|
| WT | 111 | 44 |
| Variant | * | 1 |

There is only one possible test here, the existence of the variant allele. The group sizes are 111 (71%) and 45 (29%). As Ntest=1, the significance threshold is 0.05/(202×1)=2.475×10⁻⁴. The results for this test are summarized in Table 2 with odds ratio (OR) of =4.43.

TABLE 2
Allelic correlation to treatment response (CD6 gene)

| Response | Good | None |
|---|---|---|
| No variant Allele | 37 | 74 |
| Has variant Allele | 31 | 14 |

The Fisher exact test p-value for this table is 7.211×10⁻⁵, which is better than the significance threshold.

### Example 2

### STXBP6 Markers: CGEN-40003

The amplicon identified within the STXBP6 gene is multi-allelic. The alleles for CGEN-40003 were measured using fragment analysis, and they were divided into seven groups to take into account measurement error of up to 2 bases. The relation between the allele groups and the number of TG repeats is explained in Table 3. The genotype distribution is summarized in Table 4.

TABLE 3
Allele Groups of the STXBP6 amplicon

| Allele group | Alleles by number of TG repeats |
|---|---|
| G1 | 6-7 TG repeats (SEQ ID NOs: 4-5) |
| G2 | 15-17 TG repeats (SEQ ID NOs: 13-15) |
| G3 | 18-19 TG repeats (SEQ ID NOs: 16-17) |
| G4 | 20-21 TG repeats SEQ ID NOs: 18-19) |
| G5 | 22-23 TG repeats (SEQ ID NOs: 20-21) |
| G6 | 24-25 TG repeats (SEQ ID NOs: 22-23) |
| G7 | 26-29 TG repeats (SEQ ID NOs: 24-27) |

TABLE 4
Genotype distribution of the STXBP6 amplicon

| Allele Length | G1 | G2 | G3 | G4 | G5 | G6 | G7 |
|---|---|---|---|---|---|---|---|
| G1 | 0 | 1 | 0 | 0 | 0 | 0 | 0 |
| G2 | * | 79 | 0 | 4 | 19 | 33 | 3 |
| G3 | * | * | 0 | 1 | 1 | 2 | 1 |
| G4 | * | * | * | 1 | 0 | 4 | 0 |
| G5 | * | * | * | * | 1 | 3 | 0 |
| G6 | * | * | * | * | * | 3 | 0 |
| G7 | * | * | * | * | * | * | 0 |

There are eight tests possible here (Ntest=8), imposing the 10% group size condition:

Allele groups G1+G2, recessive, group sizes 80 and 76

Allele groups G1+G2+G3+G4, recessive, group sizes 86 and 70

Allele groups G1+G2+G3+G4+G5, recessive, group sizes 107 and 49

Allele groups G1+G2, dominant, group sizes 139 and 17 (11%)

Allele group G2, recessive, group sizes 79 and 77

Allele group G2, dominant, group sizes 139 and 17

Allele group G5, dominant, group sizes 24 (15%) and 132

Allele group G6, dominant, group sizes 45 and 111

The significance threshold is 0.05/(202×8)=3.094×10⁻⁵. The best result achieved was for the second test, with the results shown in Table 5 with OR=4.01. Short alleles are therefore defined to be alleles in groups G1+G2+G3+G4 (up to 21 TG repeats, SEQ ID NOs:3-19), and long alleles are defined to be alleles in groups G5+G6+G7 (at least 22 TG repeats, SEQ ID NOs:20-28).

TABLE 5
Allelic correlation to treatment response (STXBP6 gene)

| Response | Good | None |
|---|---|---|
| Two short alleles | 50 | 36 |
| At least one long allele | 18 | 52 |

The Fisher exact test p-value for this table is 5.067×10⁻⁵, higher than the significance threshold. However, the eight tests are not independent. Inspection showed immediately that tests #4 and #6 are identical and tests #1 and #5 are almost identical. A simulation to assess the actual probability to get such a p-value as a best result of these 8 tests was therefore run. In each round the response parameter was randomly permuted between the samples and the tests were performed, recording the minimal p-value attained. After 600,000 rounds there were 83 results lower than or equal to 5.067×10⁻⁵, corresponding to an effective Ntest of 2.73 (=[83/600000]/5.067×10⁻⁵). The adjusted threshold is then 0.05/(202×2.73)=9.067×10, and thus the p-value is better.

### Example 3

### The Effect of Samples Obtained from Subjects with Moderate Response on Genotype Distribution

CGEN-40002

The distribution of genotypes for the samples obtained from subjects showing moderate response was very similar to the distribution obtained for samples of subjects showing no response. There were 67 samples without the variant allele and 12 with the variant allele (two samples had no genotype available). The results of comparing the good responders to the combined moderate and non responders are described in Table 6, with OR=4.54.

TABLE 6
Allelic correlation to treatment response (CD6 gene)

| Response | Good | Moderate + None |
|---|---|---|
| No variant allele | 37 | 141 |
| Has variant allele | 31 | 26 |

The Fisher exact test p-value for this table is 3.336×10⁻⁶, significantly better than the test performed without the moderate response samples. These results are the best obtained for all the tests, including those that added the moderate response samples to either samples obtained from the good or the non responders. If the Ntest is set to 3 (no samples of moderate responders, samples of moderate responders added to samples of good responders, samples of moderate responders added to samples of non-responders), the significance threshold is 8.251×10⁻⁵.

A total of 68 of 237 (28.7%) patients in this set had a good response to treatment. In the subset of patients with no CGEN-40002 variant allele the response was good for 37 of 178 (20.8%) and in the subset having at least one variant allele 31 of 57 (54.4%) had good response. Having at least one CGEN-40002 variant allele increases the probability of a good response to treatment significantly, while having no insertion allele lowers this probability.

CGEN-40003

Adding the samples obtained from subjects with moderate response to either the samples obtained from good or non responders resulted in inferior p-values in the case of CGEN-40003. The reason is that the genotypic distribution for the moderate responders is in the middle between the two extreme groups (50 had two short alleles and 31 had at least one long allele). The genotypic distributions may suggest a quantitative response effect, as presented in FIG. 2.

A total of 68 of 237 (28.7%) patients in this set had a good response to treatment. In the subset of patients with two CGEN-40003 short alleles the response was good for 50 of 136 (36.8%) and in the subset having at least one long allele 18 of 101 (17.8%) had good response. Having two CGEN-40003 short alleles increases the probability of a good response to treatment, while having at least one long allele lowers this probability significantly.

### Example 4

### Use of Marker Combination

As described hereinabove, two markers were identified for better response to anti-TNFα treatment, which can be summarized as (a) CGEN-40002 amplicon (in CD6 gene) has at least one variant allele comprising an insertion resulting in a 512 base pair long product, and (b) CGEN-40003 amplicon (in STXBP6 gene) has two short alleles with 21 or less TG repeats.

There are two non-trivial ways to combine the above-described two basic tests, using either conjunction or disjunction. Those combined tests, presented below, are performed for samples obtained from good responders vs. samples from non-responders; in this case both basic tests involve the same set of 156 patients.

### Conjunctive Combined Test (“CGEN-40002 has 512” and “CGEN-40003 Two Short Alleles”)

Table 7 presents the results for the conjunctive combined test

TABLE 7
Results of the conjunctive combined test

| Response | Good | None |
|---|---|---|
| Test Positive | 23 | 6 |
| Test Negative | 45 | 82 |

(OR=6.99). The Fisher exact test p-value=2.074×10⁻⁵, which is more significant than for the two basic tests; however, this is not surprising given that both of these tests are significant (at least naively).

### Disjunctive Combined Test (“CGEN-40002 has 512” or “CGEN-40003 Two Short Alleles”)

Table 8 presents the results for the disjunctive combined test:

TABLE 8
Results of the disjunctive combined test

| Response | Good | None |
|---|---|---|
| Test Positive | 58 | 44 |
| Test Negative | 10 | 44 |

(OR=5.8). The Fisher exact p-value=3.623×10⁻⁶.

The two basic tests are not correlated, as is seen by the fact that, for the two combination tests that can be devised from them, the actual results and those expected from independence of the basic tests are almost identical. This implies that, if the two basic tests are indeed valid, those combined tests would have higher odds ratios, as indeed was seen.

Moreover, the two combined tests operate in different regimes. The disjunctive combined test has the most probable outcome (“positive”) with poor separation between good and non-responders, but a minor outcome (“negative”) strongly suggesting no response. In contradistinction, the conjunctive combined test's minor outcome (“positive”) strongly suggests good response.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for predicting responsiveness to anti-TNFα therapy in a subject having or at risk of developing rheumatoid arthritis (RA), comprising determining in a biological sample obtained from the subject the allelic configuration of at least one of CD6 gene; STXBP6 gene; or a combination thereof, wherein the allelic configuration is determined by analyzing the genetic material in the biological sample for the presence of CD6 gene variant allele having an insertion of nucleic acids and/or a wild-type allele lacking the insertion; and/or for the presence of STXBP6 gene multiple short and/or long alleles having between 5 and 30 TG repeats.

2. The method of claim 1, wherein the insertion of the CD6 variant allele is at position 60,542,552 on chromosome 11 (NCBI build 36).

3. The method of claim 2 wherein the insertion is of 19 base pairs.

4. The method of claim 3, wherein the nucleic acid sequence of the CD6 variant allele comprises the nucleic acid sequence set forth in SEQ ID NO:2 and wherein the nucleic acid sequence of the wild type allele comprises the nucleic acid sequence set forth in SEQ ID NO:1.

5. (canceled)

6. The method of claim 1, wherein the STXBP6 gene configuration comprises two alleles selected from a set of short alleles comprising from 5 to 21 TG repeats and a set of long alleles comprising from 22 to 30 TG repeats.

7. The method of claim 6, wherein the TG repeats are replacing a segment located at positions 24,503,694-24,503,727 on chromosome 14 (NCBI build 36).

8. The method of claim 6, wherein the nucleic acid sequence of the short allele comprises the nucleic acid sequence set forth in any one of SEQ ID NOs:3-19 and wherein the nucleic acid sequence of the long allele comprises the nucleic acid sequence set forth in any one of SEQ ID NOs:20-28.

9. (canceled)

10. The method of claim 6, wherein the presence of an allelic configuration selected from at least one CD6 variant allele, two STXBP6 short alleles or a combination thereof is indicative of good responsiveness to anti-TNFα-treatment for RA.

11. The method of claim 6, wherein the presence of an allelic configuration selected from two CD6 wild type alleles, at least one long STXBP6 allele or a combination thereof is indicative of moderate or non-responsiveness to anti-TNFα-treatment for RA.

12. A method for determining the effectiveness of anti-TNFα therapy in a population of subjects having, or at risk of developing rheumatoid arthritis (RA), comprising: determining in a biological sample obtained from each of the subjects of the population the allelic configuration of at least one of CD6 gene; STXBP6 gene; or a combination thereof; selecting subjects having an allelic configuration selected from at least one CD6 variant allele; at least two short STXBP6 alleles or a combination thereof; determining the RA status of the selected subjects; administering the anti-TNFα therapy at least once to said subject; and determining the RA status after administration of said therapy; wherein improvement in the RA status is indicative of a positive effect of said anti-TNFα therapy.

13. The method of claim 12, wherein the nucleic acid sequence of the CD6 variant allele comprises the nucleic acid sequence set forth in SEQ ID NO:2 and wherein the nucleic acid sequence of the CD6 wild type allele comprises the nucleic acid sequence set forth in SEQ ID NO:1.

14. (canceled)

15. The method of claim 13, wherein the nucleic acid sequence of the STXBP6 short allele comprises the nucleic acid sequence set forth in any one of SEQ ID NOs:3-19 and wherein the nucleic acid sequence of the STXBP6 long allele comprises the nucleic acid sequence set forth in any one of SEQ ID NOs:20-28.

16. (canceled)

17. A kit comprising at least one oligonucleotide capable of specifically detecting the allelic configuration of at least one of CD6 gene and STXBP6 gene and reagents for employing an assay for detecting the allelic configuration of the at least one of the CD6 gene and the STXBP6 gene.

18. The kit of claim 17, wherein the allelic configuration of the CD6 gene comprises a variant allele having an insertion of nucleic acids and/or a wild-type allele lacking the insertion.

19. The kit of claim 17, wherein the allelic configuration of the STXBP6 gene comprises two alleles selected from a set of short alleles comprising from 5 to 21 TG repeats; a set of long alleles comprising from 22 to 30 TG repeats or a combination thereof.

20. The kit of claim 18, wherein the oligonucleotide is capable of specifically distinguishing between the CD6 variant and wild-type alleles.

21. The kit of claim 19, wherein the oligonucleotide is capable of distinguishing between the STXBP6 short and long alleles.

22. The kit of claim 17, wherein the oligonucleotide specifically hybridizes to a polynucleotide having a nucleic acid sequence selected from the group consisting of SEQ ID NOs:1-28.

23. The kit of claim 17, said kit comprises a primer pair capable of specifically amplifying a polynucleotide having a nucleic acids sequence selected from the group consisting of SEQ ID NOs:1-28.

24. The kit of claim 23, said kit comprises a primer pair comprising a pair of oligonucleotide having the nucleic acid sequence selected from the group consisting of SEQ ID NOs: 29-30 and 32-33.

25. (canceled)

26. The kit of claim 17, wherein the reagents are for genotyping analysis.

27. The kit of claim 26, wherein the genotyping analysis is performed employing a NAT-based assays selected from the group consisting of PCR, Real-Time PCR, LCR, Tetra-primer ARMS-PCR, Cycling Probe Reaction, Multiplex ligation-dependent probe amplification (MLPA), Fragment analysis, Dynamic allele-specific hybridization (DASH), molecular beacons, Branched DNA, RFLP analysis, Single-Strand Conformation Polymorphism analysis, Dideoxy Fingerprinting, Microarrays, Fluorescent in situ Hybridization, Comparative Genomic Hybridization, Flap endonuclease (FEN)-based assays, Invader assay, Serial Invasive Signal Amplification Reaction (SISAR), primer extension assays, 5′-nuclease activity used in Taqman assay, oligonucleotide ligase assay, temperature gradient gel electrophoresis (DGGE/TGGE), temperature gradient capillary electrophoresis (TGCE), denaturing high performance liquid chromatography (DHPLC), and High-Resolution Melting.
